(19) United States
(12) Patent Application Publication
(10) Pub. No.: US 2020/0038308 A1
(43) Pub. Date: Feb. 6, 2020

(54) WRINKLE REDUCING COMPOSITIONS AND METHODS

(71) Applicant: Kate Somerville Skincare, LLC, Beverly Hills, CA (US)

(72) Inventors: Kate Somerville, West Hollywood, CA (US); Fred Khoury, Chatsworth, CA (US)

(73) Assignee: Kate Somerville Skincare, LLC, Beverly Hills, CA (US)

(21) Appl. No.: 16/533,667

(22) Filed: Aug. 6, 2019

Related U.S. Application Data
(60) Provisional application No. 62/715,051, filed on Aug. 6, 2018.

(51) Int. Cl. A61K 8/73; A61K 8/88; A61K 8/06; A61Q 19/08; A61K 31/728; A61K 31/198; A61K 9/107; A61P 17/00

(52) U.S. Cl. CPC A61K 8/735; A61K 8/88; A61K 8/066; A61P 17/00; A61K 31/728; A61K 31/198; A61K 9/107; A61Q 19/08

(57) ABSTRACT

Described is a cosmeceutical formulation for treating damaged skin comprising a first hyaluronic acid having a molecular weight of from about 25 kDa to about 100 kDa, a second hyaluronic acid having a molecular weight of from about 1 kDa to about 20 kDa, and a polyglutamate. The formulation is used in methods of improving skin, including treating or repairing damaged or wrinkled skin.

This application claims the benefit of the filing date of U.S. Provisional Application No. 62/715,051, filed Aug. 6, 2018, which is incorporated by reference herein in its entirety.

### FIELD OF THE INVENTION

This invention relates to cosmeceutical compositions and methods for administration to treat or repair damaged or wrinkled skin. In particular, the cosmeceutical compositions comprises at least two hyaluronic acids having different molecular weights and a polyglutamate. The invention also relates to use of the composition for treating damaged or wrinkled skin.

### BACKGROUND INFORMATION

Aging and UV-B radiation from the sun can deteriorate and damage the skin. For example, properties of the skin such as degrees of hydration, tightness, firmness, elasticity, texture, and smoothness can be adversely affected by age and sun. In addition, sun and aging can cause a variety of wrinkles and lines to appear on the skin, particularly on the face. There is a continuing need for safe and effective treatments to improve the quality and appearance of the skin.

### SUMMARY OF THE INVENTION

A cosmeceutical formulation for treating damaged skin is provided that includes a first hyaluronic acid having a molecular weight of from about 25 kDa to about 100 kDa, a second hyaluronic acid having a molecular weight of from about 1 kDa to about 20 kDa, and a polyglutamate. The cosmeceutical formulation may further include a third hyaluronic acid having a molecular weight of greater than about 500 kDa. Also described is a method for treating damaged or wrinkled skin by administering a cosmeceutical formulation comprising a first hyaluronic acid having a molecular weight of from about 25 kDa to about 100 kDa, a second hyaluronic acid having a molecular weight of from about 1 kDa to about 20 kDa, and a polyglutamate. A method for improving skin is provided, by administering a cosmeceutical formulation comprising a first hyaluronic acid having a molecular weight of from about 25 kDa to about 100 kDa, a second hyaluronic acid having a molecular weight of from about 1 kDa to about 20 kDa, and a polyglutamate.

### DESCRIPTION OF EMBODIMENTS OF THE INVENTION

The present inventors have found, surprisingly, that a combination of at least two hyaluronic acids having different molecular weights and polyglutamate results in dermal rejuvenation, including significant effects on damaged skin, such as hydration benefits, skin plumping and line and wrinkle reduction. The formulations are cosmeceuticals, i.e. suitable for topical administration to a subject, with cosmetically and/or pharmaceutically acceptable carriers and components, and provide cosmetic and/or pharmaceutical effects. One aspect of the invention is a cosmeceutical compositions for treating damaged or wrinkled skin. Surprisingly, the use of a combination of a first hyaluronic acid, a second hyaluronic acid having different molecular weights, and polyglutamate provides enhanced improvement in skin properties and wrinkle reduction than what would be expected by individually applying the three components. In embodiments, the first hyaluronic acid has a low molecular weight and the second hyaluronic acid has a very low molecular weight as further identified and described herein.

In the description and examples that follow, all temperatures are set forth in uncorrected degrees Celsius. Unless otherwise indicated, all parts and percentages are by weight. As used herein, the term “about” refers to plus or minus 10% of the indicated value. As used herein, the singular forms “a,” “an,” and “the” include plural referents unless the context clearly dictates otherwise.

As used herein, the terms “treatment” and “repair” of damaged and wrinkled skin and equivalent expressions thereof, means to at least improve the appearance of skin by reducing the presence of wrinkling or improve a physical characteristic of the skin. Physical characteristics include the degree of hydration, tightness, firmness, elasticity, texture, and smoothness. Wrinkling includes crow's feet fine lines, crow's feet wrinkles, fine lines, deep wrinkles, forehead wrinkles, eleven lines, marionette lines, parentheses lines, and eye area lines.

### Hyaluronic Acid (HA)

HA is a skin hydrating agent that can help restore water to dehydrated skin. When applied according to a method of the invention, the HA delivers substantially instant hydration to the skin.

HA is a naturally occurring substance found in various connective tissue in the human body. HA is a non-sulfated glycosaminoglycan, naturally found in the human body and is the main component of the extracellular matrix. HA is found in high levels in the skin, where it is naturally produced by both fibroblasts and keratinocytes and exists as a polymer of medium molecular weight (500 kDa-2,000 kDa). An important function of HA is to hold water in the intercellular matrix of the connective tissue. This water-binding capacity significantly contributes to the elasticity of the skin, serving as a water reservoir. With aging and UV-B damage, the quantity and quality of hyaluronic acid in the skin decreases, which leads a loss of elasticity and the increase of wrinkles.

HA used in formulations of the invention can be any of a variety of forms that will be evident to a skilled worker. These include, for example, salts (such as the sodium salt, sodium hyaluronate) and derivatives that will be evident to a skilled worker. Thus, as used herein, the terms “hyaluronic acid,” “HA,” and “hyaluraonate,” refer not only to the acid, but also to salts such as sodium hyaluronate, and the terms are used interchangeably, unless dictated otherwise by context. Unless specified otherwise, sodium hyaluronate can be can be replaced with other hyaluronic salts such as potassium hyaluronate and the like. In general, hyaluronic acid and hyaluronate salts can be used interchangeably in products and formulations of the invention, unless stated otherwise. Persons skilled in the art will be able to identify appropriate forms of hyaluronic acid.

The hyaluronic acid used in the present invention can be obtained from a natural source or can be a synthetic bioidentical hyaluronic acid that is indistinguishable from naturally occurring hyaluronic acid. The hyaluronic acids of different molecular weights used in the present invention can be prepared synthetically or from a higher molecular weight hyaluronic acid by chemical hydrolysis or enzymatic hydrolysis, for example, hydrolysis by hyaluronidase.

Cosmeceutical compositions of the invention include a first hyaluronic acid having a low molecular weight, for example, from about 25 kDa to about 100 kDa, referred to herein as “low molecular weight HA,” “low MW HA,” or “LMWHA.” Low MW HA may be prepared synthetically or can be a hydrolyzed hyaluronic acid prepared from a higher molecular weight HA. In any embodiment, the low MW HA can have a molecular weight of about 50 kDa or less, or about 50 kDa. The low MW HA allows for increased permeation through the skin compared to higher molecular weight hyaluronic acid. The low MW HA can assist in rejuvenating the skin by improving viscoelastic properties and can significantly decrease deep wrinkles. Low MW HA is commercially available from a number of sources; for example, as sold under the brand name HyaCare® 50, available from Evonik Industries (Essen, Germany). In embodiments, the first hyaluronic may be provided in an amount of from about 0.001 wt % to about 0.500 wt % of the total formulation, from about 0.025 wt % to about 0.300 wt % of the total formulation, or about 0.100 wt % of the total formulation.

Cosmeceutical compositions of the invention also include a second hyaluronic acid having a very low molecular weight, for example, from about 1 kDa to about 20 kDa, referred to herein as “very low molecular weight HA,” “very low MW HA,” or “VLMWHA.” Very low MW HA may be prepared synthetically or can be a hydrolyzed hyaluronic acid prepared from a higher molecular weight HA. In any embodiment, the very low MW HA can have a molecular weight of about 10 kDa or less, or from about 3 kDa to about 10 kDa. It has been found that very low MW HA provides a deeper penetration than the first HA or the third HA (described below), achieving, for example, increased hydration of the skin and reduction of a wide range of wrinkles. The second HA is commercially available from a number of sources and has been referred to as “Oligo HA,” and, for example, sold under the brand name miniHA™, available from Bloomage Freda BioPharm (Jinan, P.R. China). In embodiments, the second hyaluronic may be provided in an amount of from about 0.001 wt % to about 0.500 wt % of the total formulation, from about 0.025 wt % to about 0.300 wt % of the total formulation, or about 0.050 wt % of the total formulation.

Any embodiment of the invention can also include a third hyaluronic acid, which can be a naturally occurring HA or its synthetic equivalent. As such, the third hyaluronic acid has a higher molecular weight. In embodiments, the third HA has a molecular weight of from about 500 kDa to about 2,000 kDa, or from about 600 kDa to about 1000 kDa. The third HA may be provided as a pure HA or as a bio-derived HA together with other ingredients derived from a natural source. When added as a pure HA, the third HA may be present in an amount of from about 0.0001 wt % to about 5 wt %, from about 0.01 wt % to about 3 wt %, or from about 0.01 wt % to about 0.05 wt %. An exemplary material that includes bio-derived HA is commercially available as MariMoist® from BioCogent, LLC (Stony Brook, N.Y.). In embodiments, MariMoist® may be provided in an amount of from about 0.01 wt % to about 30 wt % of the total formulation, from about 0.100 wt % to about 5 wt % of the total formulation, or about 1.0 wt % of the total formulation. It is believed that MariMoist contains from about 0.01 to about 0.1 natural HA. Thus, in some embodiments, the third HA may be provided from about 0.000001 wt % to about 0.03 wt % of the total formulation, from about 0.00001 wt % to about 0.005 wt % of the total formulation, or about 0.0001 wt % to about 0.001% wt %.

### Polyglutamate (PGA)

Polyglutamic acid (PGA) is a polymer of glutamic acid. It can be naturally occurring, in which case it may be produced by bacterial fermentation. Synthetic PGA typically has a molecular mass of less than 10 kDa. Bacterially produced PGA can have a molecular mass of greater than 10 kDa, for example ranging from about 100 kDa to about 1000 kDa, and can be higher. In any embodiment of the invention, the polyglutamate can include a polyglutamate having an average molecular weight of about 2600 kDa. According to embodiments, the polyglutamate can also or alternatively include a polyglutamate having an average molecular weight of about 1000 kDa. In some embodiments, the polyglutamate may include a mixture of one or more polyglutamates having different molecular weights. According to embodiments, the polyglutamate may include a mixture of a first polyglutamate having an average molecular weight of about 1000 kDa, and a second polyglutamate having an average molecular weight of about 2600 kDa. PGA can enhance moisture binding and retention in the skin, essentially assisting the function of HA. In addition, PGA has been found to inhibit enzymatic hydrolysis of HA, thus maintaining HA levels in the skin and increasing the effectiveness of HA.

PGA used in a formulations of the invention can be any of a variety of forms that will be evident to a skilled worker. These include, for example, salts (such as the sodium salt, sodium polyglutamate) and derivatives that will be evident to a skilled worker. Thus, as used herein, the terms “polyglutamic acid,” “PGA,” and “polyglutamate,” refer not only to the acid, but also to salts such as sodium hyaluronate, and the terms are used interchangeably, unless dictated otherwise by context. Unless specified otherwise, sodium polyglutamate can be can be replaced with other polyglutamate salts such as potassium polyglutamate and the like. In general, polyglutamate acid and polyglutamate salts can be used interchangeably in products and formulations of the invention, unless stated otherwise. Persons skilled in the art will be able to identify appropriate forms of polyglutamic acid.

In embodiments of the invention, PGA may be provided as single component or together with other ingredients. Embodiments of the invention can include a first PGA with a molecular weight of, for example, from about 500 kDa to about 2000 kDa, from about 750 kDa to about 1500 kDa, or about 1000 kDa. The first PGA may be present in an amount of from about 0.001 wt % to about 2 wt % of the total formulation, from about 0.1 wt % to about 5 wt % of the total formulation, or about 0.05 wt % of the total formulation. Embodiments of the invention can include a second PGA with a molecular weight of, for example, from about 1000 kDa to about 5000 kDa, from about 2000 kDa to about 3000 kDa, or about 2600 kDa. An exemplary pure PGA used in embodiments of the invention is sodium polyglutamate commercially available as Hyafactor™-PGA (HMW), available from Bloomage Freda BioPharm (Jinan, P.R. China). An exemplary PGA sold as a mixture is MegaMoist 2MKD, available from BC Research Company Inc. (Elmwood Park, N.J.).

MegaMoist 2MKD is believed to be a mixture of Water, Propanediol, Polyglutamic Acid, and Phenoxyethanol. The PGA in MegaMoist 2MKD has a molecular weight of about 2600 kDa and comprises about 0.5 wt %. In embodiments, MegaMoist 2MKD may be provided in an amount of from about 0.01 wt % to about 15 wt % of the total formulation, from about 0.1 wt % to about 5 wt % of the total formulation, or about 3 wt % of the total formulation. Thus, in embodiments, the second polyglutamate may be provided in an amount of from about 0.00005 wt % to about 0.075 wt % of the total formulation, from about 0.0005 wt % to about 0.025 wt % of the total formulation, or about 0.015 wt % of the total formulation.

Table 1 identifies exemplary amounts of the various HA and polyglutamate in compositions according to the invention.

TABLE 1
HA and polyglutamate content in exemplary
embodiments of the invention.

| | Natural HA | LMWHA | VLMWHA | Polyglutamate |
|---|---|---|---|---|
| | (wt %)^{a} | (wt %) | (wt %) | (wt %)^{b} |
| Formula 1 | | 0.001-5 | 0.001-5 | 0.005-5 |
| Formula 2 | 0.0001-5 | 0.001-0.5 | 0.001-0.5 | 0.001-2 |
| | | | | 0.00005-0.75 |
| Formula 3 | 0.01-3 | 0.025-0.3 | 0.025-0.3 | 0.025-0.5 |
| | | | | 0.0005-0.025 |
| Formula 4 | 0.01-0.05 | 0.1 | 0.05 | 0.0153 |

^{a}Quantity represents total amount of extract of Chondrus crispus in combination with bio-derived HA.
^{b}When two values are present, the top number represents the amount of the first polyglutamate and the bottom number represents an amount of the second polyglutamate.

The amount of polyglutamates in Table 1 may be a mixture of the first and second polyglutamates (as shown in Formulas 2-4) or may be include only the first polyglutamate or only the second polyglutamate in the total amount shown.

### Additional Components

Any embodiment of the invention can include perfluorocarbons. The use and content of perfluorocarbons in skin treatment cosmeceutical compositions is disclosed in U.S. Pat. No. 8,980,227, which is incorporated herein by reference in its entirety. However, embodiments of the invention can include lower amounts of perfluorocarbons than described in U.S. Pat. No. 8,980,227. The perfluorocarbons used in the invention can be, for example, one or more of perfluoropropane, perfluorobutane, perfluoropentane, perfluorohexane, perfluorodecalin, perfluorodimethyl-cyclohexane, perfluoroperhydrophenanthrene, pentafluoro-propane, perfluorotripropylamine, C6-C9 perfluoroalkanes, perfluoroperhydrofluoranthrene, perfluorodecalin, perfluoroperhydrophenanthrene, bis(perfluor-hexyl)-1,2-ethene, perfluoro-1,3-dimethylcyclohexane, perfluoromethyldecalin, perfluoroisopropyldecalin, a mixture of perfluorodixylylmethane and perfluorodixylylethane, and/or a mixture of perfluoroperhydrophenanthrene and perfluoro n-butyldecalin. In exemplary embodiments, a mixture of at least three different perfluorocarbons is used. In embodiments, the total amount of perfluorocarbons may be from about 0.005 wt % to about 30 wt % of the total formulation, from about 1 wt % to about 10 wt % of the total formulation, or about 5 wt % of the total formulation. It was found that the present invention achieves the benefits of perfluorocarbons when used in lower amounts than typically exemplified in U.S. Pat. No. 8,980,227.

In some embodiments, the cosmeceutical formulation of the invention may be packaged in a pressurized container for dispensing. In that configuration, a suitable propellant may be added before or after the composition is packaged. Exemplary propellants include, for example, one or more liquified hydrocarbons of 1 to 10 carbon atoms, such as n-propane, n-butane, isobutene, isopentane, or n-pentane, an ether (e.g., dimethyl ether), nitrogen, compressed air, or other inert gas, and mixtures thereof. Propellants, when added, are typically used in an amount of from about 5 to about 20% of the formulation.

In embodiments of the cosmeceutical formulation of the invention, the formulation can be an aqueous formulation, an oil and water emulsion (e.g., containing about 60 wt %-90 wt % purified water and about 10 wt % to about 40 wt % components forming an oil phase). “Purified water” is water that does not contain ingredients which would be harmful to, or would cause adverse reactions to, the skin of a subject, such as a human. Distilled water and/or deionized water can be used.

In addition to the components noted above, embodiments of a cosmeceutical formulation of the invention can include one or more additional components. Additional components include, for example, Vitamins (for example, niacin, vitamin E, vitamin C), minerals, enzymes, amino acids, antioxidants, and fragrances.

Any embodiment of the invention can include ingredients and components known to be useful in the manufacture of cosmetic compositions. The components can include (with approximate range of composition wt % indicated in parentheses): Emulsifiers (0.1-20%), such as nonionic, cationic, anionic or polymeric emulsifiers, (e.g., glyceryl stearate, cetearyl alcohol, cetearyl phosphate, behentrimonium chloride, polysorbate-20, acrylaytes/C10-30 alkyl acrylate crosspolymer, etc); Rheology modifiers (0.05-5%) (e.g., polyacrylic acid polymers, xanthan gum, cellulose gums, silicates, alginates, hydrocolloids); Humectants (0.5-8%) (e.g., propanediol, 1,2-hexandiol, glycerin and other glycols, including butylene glycol); Surfactants (0.1-5%), including, non-ionic, cationic and anionic surfactants (e.g., ethoxylated and non-ethoxylated, and amino acid surfactants, including caprylhydroxamic acid, polysorbate 20, sorbitan monooleate, sodium lauroyl glutamate, laureth-12); Emollients (0.1-20%) (e.g, squalane, ethylhexyl palmitate, diisopropyl dimer dilinoleate, C12-15 Alkyl Benzoate, Melalenca alternifolia (Tea Tree Leaf), trimethylolpropane tricaprylate/tricaprate, isosobide dicaprylate, oleyl alcohol); pH modifiers and buffers (0.005-2%) (e.g., triethanolamine, sodium hydroxide, acidifiers, including citric acid); Antimicrobial agents (0.001-3%), (e.g., salicylic acid, or preservatives, such as, e.g., phenoxyethanol, benzyl alcohol, or potassium sorbate); Aromas including fruit or plant extracts (0.005-5%), for example in the form of fragrances or essential oils; (e.g., Zanthoxylum Bungeanum fruit extract, Echinacea purpurea extract, chondrus crispus extract, hydrolyzed chondrus crispus extract, tropaeolum majus flower/leaf/stem extract, or lavandula angustilfolia (lavender) flower oil); Antioxidants (e.g., ascorbic acid or derivatives thereof, including ascorbyl palmitate, ascorbyl glucoside, ascorbyl isostearate, sodium ascorbyl phosphate, magnesium ascorbyl phosphate, ethyl ascorbic acid, or aminopropyl ascorbyl phosphate; or tocopherol of derivatives thereof, including tocopheryl acetate, tocopheryl oleate, or tocopheryl linoleate); additional skin care antiaging/anti-wrinkle agents (0.005-5%, e.g. 0.5-1%), (e.g., licorice extract and derivatives, retinol, adenosine, or beatine); Film-forming agents (0.005-5%, e.g. 0.5-1%) (e.g., polysilicone-11); and FD&C colors (0.001-30%), for example, iron oxides, titanium dioxides, including silicon-treated pigments, in the form of powder particle or pre-dispersions in various dispersants, such as castor oil, silanetriol, silicone, hydroxystearic Acid, mineral oils, or C₁₂-C₁₅ alkyl benzoate).

Tables 2-4 identify exemplary formulations according to the invention that include ingredients in addition to the various hyaluronic acids and polyglutamate. Persons skilled in the art will recognize that certain components, for example emollients such as dimethicone and trimethylolpropane tricaprylate/tricaprate, can be replaced by other components with similar function. In most cases, these can be used interchangeably and persons skilled in the art of cosmetic and cosmeceutical formulations can adjust amounts to achieve a desired consistency and feel in the product. However, the inventors have found that the formulations described below achieve particularly desirable results. In particular, the presence and amounts of HA and PGA provide a superior product. The presence and amounts of HA and PGA provide a synergistic result, in which the inventors believe may be achieved by the reduction of hyaluronidase, an enzyme responsible for the degradation of hyaluronic acid. The combination of HA and PGA in accordance with the invention provides the skin benefit of HA, while also reducing the breakdown of the skin's own HA.

Particularly advantageous ingredients that can be included in formulations according to the present invention include extracts of Zanthoxylum bungeanum, which has an effect similar to botox, intracellular oxygen boosters, and Echinacea extract. Zanthalene (available from Indena (Seattle, Wash.)), is an exemplary extract from the fruit of Zanthoxylum bungeanum (Sichuan pepper), and provides an additional lifting effect to the skin. Intracellular oxygen boosters, include, for example, Cerasome Oxygen (a ceramide based delivery system containing molecular oxygen which is distributed by ROVI cosmetics), or Tropaeolum Majus Flower/Leaf/Stem Extract (for example as marketed as Oxygeskin by Silab (Saint-Viance, FR)). An exemplary Echinacea extract is Volpura (available from Biocogent), which is a mixture of propanediol and Echinacea Purpurea.

TABLE 2
Exemplary Formulation A

| Component | Wt. % |
|---|---|
| Purified Water | 50-90% |
| Perfluorocarbons (e.g., Perfluorohexane, perfluoro- | 0 to 10% |
| perhydrophenanthrene, perfluorodecalin, perfluoro- | |
| dimethylcycloheane) | |
| Low Molecular weight Hyaluronic Acid (e.g., about | 0.005 to 5% |
| 50 kDa) | |
| Very Lowe Molecular Weight HA (e.g., 3-10 kDa) | 0.005 to 5% |
| Emulsifiers (Non-ionic Cationic, Anionic & poly- | 0.1 to 20% |
| meric (including, e.g., glyceryl stearate, cetearyl | |
| alcohol, Cetearyl Phosphate, Behentrimonium Chloride, | |
| Acrylates/C10-30 Alkyl Acrylate Crosspolymer, etc.)) | |
| Rheology modifiers (e.g., polyacrylic acid polymers, | 0.05 to 5% |
| xanthan gum, cellulose gums, silicates, alginates, | |
| hydrocolloids, etc.) | |
| Humectant (e.g., propanediol, glycerin & other | 0.5 to 5% |
| glycols) | |
| Surfactants (e.g., Nonionic, Cationic and Anionic, | 0.1 to 5% |
| Ethoxylated and Non Ethoxylated surfactancts) | |
| Emollients | 0.1 to 20% |
| Antioxidants | 0.005 to 5% |
| pH modifier | 0.005 to 2% |
| Antimicrobial agents (e.g., salicylic acid, and | 0.001 to 3% |
| preservatives) | |
| Colorants (e.g., FD& C colors, silicon-treated | 0.001 to 30% |
| pigments) | |
| Aroma (e.g., fragrance or essential oils) | 0.005 to 5% |
| Polyglutamate | 0.005-15% |
| Propellant (e.g., liquefied hydrocarbon of 1 to 10 | 5-20% |
| carbon atoms, such as n-propane, n-butane, isobutene, | |
| isopentane, n-pentane and mixtures thereof; and/or | |
| ethers (dimethyl ether), nitrogen or compressed air) | |

TABLE 3
Exemplary Formulation B

| Component | Wt % |
|---|---|
| Purified Water | 50.000-90.000 |
| Caprylhydroxamic Acid/1,2-Hexanediol/Propanediol | 0.250-3.000 |
| Adenosine | 0.010-0.050 |
| Betaine | 0.010-10.000 |
| Glyceryl Stearate/Cetearyl Alcohol/Stearic Acid/ | 0.010-10.000 |
| Sodium Lauroyl Glutamate | |
| Trimethylolpropane Tricaprylate/Tricaprate | 0.010-10.000 |
| Dimethicone | 0.010-10.000 |
| Polysilicone-11/Laureth-12 | 0.010-5.000 |
| Isosorbide Dicaprylate | 0.010-10.000 |
| Dimethicone | 0.010-10.000 |
| Dimethicone | 0.010-10.000 |
| Oleyl alcohol/Zanthoxylum Bungeanum Fruit | 0.010-7.000 |
| Extract | |
| Acrylates/C10-30 Alkyl Acrylate Crosspolymer | 0.010-3.000 |
| Perfluorodecalin/Perfluorohexane/perfluoroperhydro- | 1.000-10.000 |
| phenanthrene | |
| Sodium Hydroxide | 0.010-3.000 |
| Propanediol/Echinacea Purpurea | 0.010-5.000 |
| Water/Chondrus Crispus Extract/Hydrolyzed Chondrus | 0.010-30.000 |
| Crispus Extract/Sodium Hyaluronate/Phenoxyeth- | |
| anol/Potassium Sorbate/Butylene Glycol/Sodium | |
| Dihydroacetate/Citric Acid | |
| Water/Glycerin/Glycine (Soybean) Soja Extract/ | 0.010-3.000 |
| Phenoxyethanol | |
| Hydrolyzed Vegetable Protein/Trehalose/Glycerin/Aqua | 0.010-10.000 |
| Water (Aqua)/Propanediol/Polyglutamic Acid/Phenoxy- | 0.010-15.000 |
| ethanol | |
| Sodium Polyglutamate | 0.001-2.000 |
| Low Molecular Weight Hyaluronic Acid | 0.001-0.500 |
| Very Low Molecular Weight Hyaluronic Acid | 0.001-0.500 |
| Tropaeolum Majus Flower/Leaf/Stem Extract | 0.001-5.000 |
| Lavandula Angustilfolia (Lavender) Flower Oil | 0.005-3.000 |
| Sodium hydroxide | 0.010-3.000 |

TABLE 4
Exemplary Formulation C

| Component | Wt % |
|---|---|
| Purified Water | 60.000-90.000 |
| Caprylhydroxamic Acid/1,2-Hexanediol/Propanediol | 1.500-2.500 |
| Adenosine | 0.040-0.045 |
| Betaine | 0.025-5.000 |
| Glyceryl Stearate/Cetearyl Alcohol/Stearic Acid/ | 0.100-5.000 |
| Sodium Lauroyl Glutamate | |
| Trimethylolpropane Tricaprylate/Tricaprate | 0.025-5.000 |
| Dimethicone | 0.025-5.000 |
| Polysilicone-11/Laureth-12 | 0.100-5.000 |
| Isosorbide Dicaprylate | 0.025-5.000 |
| Dimethicone | 0.025-5.000 |
| Dimethicone | 0.025-5.000 |
| Oleyl alcohol/Zanthoxylum Bungeanum Fruit | 0.100-3.000 |
| Extract | |
| Acrylates/C10-30 Alkyl Acrylate Crosspolymer | 0.050-0.500 |
| Perfluorodecalin/Perfluorohexane/Perfluoroperhydro- | 1.000-10.000 |
| phenanthrene | |
| Sodium Hydroxide | 0.010-3.000 |
| Propanediol/Echinacea Purpurea | 0.100-5.000 |
| Water/Chondrus Crispus Extract/Hydrolyzed Chondrus | 0.100-5.000 |
| Crispus Extract/Sodium Hyaluronate/Phenoxyeth- | |
| anol/Potassium Sorbate/Butylene Glycol/Sodium | |
| Dihydroacetate/Citric Acid | |
| Water/Glycerin/Glycine (Soybean) Soja Extract/ | 0.100-3.000 |
| Phenoxyethanol | |
| Hydrolyzed Vegetable Protein/Trehalose/Glycerin/Aqua | 0.100-5.000 |
| Water (Aqua)/Propanediol/Polyglutamic Acid/Phenoxy- | 0.100-5.000 |
| ethanol | |
| Sodium Polyglutamate | 0.025-0.500 |
| Low Molecular Weight Hyaluronic Acid | 0.025-0.300 |
| Very Low Molecular Weight Hyaluronic Acid | 0.025-0.300 |
| Tropaeolum Majus Flower/Leaf/Stem Extract | 0.100-5.000 |
| Lavandula Angustilfolia (Lavender) Flower Oil | 0.005-0.500 |
| Sodium hydroxide | 0.010-3.000 |

### Manufacturing Procedure:

Compositions according to the invention can be manufactured using standard techniques known in the art. In one embodiment, an aqueous phase is prepared containing the water and, for example, Caprylhydroxamic Acid/1,2-Hexanediol/Propanediol, Adenosine, Betaine, and Glyceryl Stearate/Cetearyl Alcohol/Stearic Acid/Sodium Lauroyl Glutamate, and mixed until all the solids are dissolved or dispersed and the batch is uniform. The aqueous phase is held and may be warmed, for example to about 75° C. An oily phase is separately prepared that may contain, for example, Trimethylolpropane Tricaprylate/Tricaprate, Dimethicone, Polysilicone-11/Laureth-12, Isosorbide Dicaprylate, Dimethicone, Oleyl alcohol/Zanthoxylum Bungeanum Fruit Extract, and Acrylates/C10-30 Alkyl Acrylate Crosspolymer. The oily phase may be warmed, for example to about 75° C. The aqueous and oily phase can then be combined and mixed, for example with a propeller mixer, and optionally homogenized until a desired consistency is reached. The combined phases can then be cooled. The pH can then be adjusted, for example using aqueous sodium hydroxide, if desired or necessary. A suitable perfluorocarbon mixture can be added followed by mixing, and the pH adjusted (for example using aqueous sodium hydroxide) if necessary. The remaining ingredients, including the natural HA, LMWHA, VLMWHA and polyglutamate, and optionally including other ingredients (for example, Propanediol/Echinacea Purpurea, Water/Glycerin/Glycine (Soybean) Soja Extract/Phenoxyethanol, Hydrolyzed Vegetable Protein/Trehalose/Glycerin/Aqua Tropaeolum Majus Flower/Leaf/Stem Extract, and Lavandula Angustilfolia (Lavender) Flower Oil), are combined and then added to the previously combined ingredients, followed by a final pH adjustment (for example using aqueous sodium hydroxide), if necessary.

After mixing, the formulation is placed in a suitable container. If the formulation is to be dispensed as a pressurized aerosol, a suitable liquid propellant is added at a desired ratio.

### Use of the Formulation

Aging and UV-B radiation from the sun can deteriorate and damage the skin. For example, properties of the skin such as degrees of hydration, tightness, firmness, elasticity, texture, and smoothness can be adversely affected by age and sun. In addition, sun and aging can cause a variety of wrinkles and lines to appear on the skin, particularly on the face. Wrinkles and fine lines include fine lines, deep wrinkles, forehead wrinkles, “eleven lines” (lines appearing between the eye brows), “marionette lines” (vertical lines from lip to chin), and “parentheses lines” (vertical lines from nose to chin).

In order to improve skin properties and repair damage, the cosmeceutical formulation of the invention is applied to the skin at least once, and preferably twice, per day. When applying two times per day, it is preferred to administer once in the morning, and once in the evening. The formulation is applied by massaging it on the skin with fingers. After application, the formulation is allowed to absorb into the skin. In embodiments of the invention, the cosmeceutical formulation is an aerosol, a serum, a foaming agent, a mist or a cream.

While a single application has been found to improve skin properties and alleviate damage, repeated use further improves results. Similarly, repeated application (daily or twice a day) extends the improvement in skin properties and reduction of wrinkles. Visible and measurable improvements in skin qualities and wrinkle reduction are observed after a single use, and increase when used twice daily use of one week and twice daily for four weeks.

Formulations of the invention as described and used herein can provide a decrease in the presence of wrinkles and improvement in skin properties. The decrease in wrinkles and improvement in properties is visually observable and can result in a quantifiable improvement. The repair may be permanent, but is more frequently temporary, lasting at least one day, or at least one week. The period of temporary repair may be extended by repeated application of the inventive formulation. The degree of repair is similarly improved upon by repeated administration.

The invention is further exemplified by the non-limiting examples that follow:

### EXAMPLE 1

### Exemplary Formulation and Method of Manufacture

An exemplary formulation with the components as shown in Table 5 below, in which wt % represents the approximate weight percent in the final formulation, was prepared as follows:
Phase A—Into the main processing tank, add DI Water, start high speed mixing. Add the remaining ingredients of Phase A, one ingredient at a time making sure to mix until uniform before adding the next. Mix until all the solids are dissolved or dispersed and the batch is uniform. Heat to 75° C. and hold.
Phase B—Into the secondary processing tank, add the ingredients of Phase B, one ingredient at a time making sure to mix until uniform before adding the next. Mix until all the solids are dissolved or dispersed and the batch is uniform. Heat to 75° C. and hold.

Add Phase B to Phase A, propeller mix for 20-30 minutes allowing polymers to fully hydrate. Homogenize for 5-10 minutes at 2500-3000 rpm, then propeller and sweep mix. Cool Batch to 35° C.
Phase C—Add Phase C to main batch and Mix until uniform.
Phase D—QS (quantum satis) pH to 5.50-5.75 with Sodium Hydroxide 50% aq sol'n. Mix until uniform.
Phase E—Add Phase E to main batch one ingredient at a time making sure to mix until uniform before adding the next. Mix until uniform.
Phase F—QS pH to 5.50-5.75 with Sodium Hydroxide 50% aqueous solution. Mix until uniform. QS batch with DI water, if necessary. Mix until uniform. Using a clean container, bring top and bottom sample and the completed.

TABLE 5
Formulation Example.

| Phase | Component | Wt. % |
|---|---|---|
| Phase A | Water | 72.175 |
| (Aqueous | Caprylhydroxamic Acid/1,2-Hexanediol/ | 2.000 |
| phase) | Propanediol | |
| | Adenosine | 0.045 |
| | Betaine | 1.000 |
| | Glyceryl Stearate/Cetearyl Alcohol/Stearic | 1.250 |
| | Acid/Sodium Lauroyl Glutamate | |
| Phase B | Trimethylolpropane Tricaprylate/Tricaprate | 2.000 |
| (Oily | Dimethicone | 0.250 |
| Phase) | Polysilicone-11/Laureth-12 | 0.500 |
| | Isosorbide Dicaprylate | 2.000 |
| | Dimethicone | 2.200 |
| | Oleyl alcohol/Zanthoxylum Bungeanum | 1.000 |
| | Fruit Extract | |
| | Acrylates/C10-30 Alkyl Acrylate Crosspolymer | 0.125 |
| Phase C | Perfluorodecalin/Perfluorohexane/Perfluoro- | 5.000 |
| | perhydrophenanthrene | |
| Phase D | Sodium Hydroxide | 0.130 |
| Phase E | Propanediol/Echinacea Purpurea | 2.000 |
| | Water/Chondrus Crispus Extract/Hydrolyzed | 1.000 |
| | Chondrus Crispus Extract/Sodium Hyaluronate/ | |
| | Phenoxyethanol/Potassium Sorbate/Butylene | |
| | Glycol/Sodium Dehydroacetate/Citric Acid | |
| | Water/Glycerin/Glycine (Soybean) Soja | 2.000 |
| | Extract/Phenoxyethanol | |
| | Hydrolyzed Vegetable Protein/Trehalose/ | 1.000 |
| | Glycerin/Aqua | |
| | Water (Aqua)/Propanediol/Polyglutamic Acid/ | 3.000 |
| | Phenoxyethanol | |
| | Sodium Polyglutamate | 0.050 |
| | Very Low Molecular Weight Hyaluronic Acid | 0.050 |
| | Low Molecular Weight Hyaluronic Acid | 0.100 |
| | Tropaeolum Majus Flower/Leaf/Stem Extract | 1.000 |
| | Lavandula Angustilfolia (Lavender) Flower Oil | 0.125 |
| Phase F | Sodium Hydroxide (pH 5.25-5.75) | 0.065 |

### EXAMPLE 2

### Consumer Perception and Clinical Evaluation of Skin Treatment Product

### 1.0 Objectives

A study was conducted to evaluate the skin treatment product of the invention and determining whether if improves skin firmness; improves skin elasticity; improves skin hydration; improves the appearance or reduces the presence of crow's feet fine lines; and improves the appearance or reduces the presence of crow's feet wrinkles in a panel of 30 female subjects, aged 35-65 years. To assess the qualitative improvements, surveys of the users' experience were conducted after 30 minutes following a single application and after 1 and 4 weeks of product use. To assess quantitative improvements, Cutometer® and Corneometer® measurements were conducted after 30 minutes following a single application and after 1 and 4 weeks of product use.

### 2.0 Test Subjects

A sufficient number of individuals, between 35 and 65 years of age (inclusive) and in general good health, were empanelled so that at least 30 successfully completed the study. All subjects were required to read, understand and sign a written Informed Consent Form and complete a brief Medical History Form.

### 3.0 Study Design

3.1 Subject Selection

Subjects were to be enrolled in accordance with the following inclusion/exclusion criteria.

3.1.1 Inclusion Criteria

1. Females between the ages of 35 and 65 years (inclusive) in general good health (no physical required).

2. Individuals with a crow's feet fine line and wrinkle score of “5” (noticeable) or greater on the face, for qualification purposes only.

3. Individuals who indicate their willingness to participate in the study, follow directions and to stay on the study for the full 4 weeks.

4. Individuals who can read, understand and sign the Informed Consent Form.

3.1.2 Exclusion Criteria

1. Women who are pregnant, planning a pregnancy, lactating and/or nursing a child.

2. Individuals with any visible skin disease.

3. Individuals with sunburn, suntan on the face or planning a vacation with sun-exposure or planning the use of a tanning booth during the course of the study.

4. Individuals engaged in a concurrent research project of a facial product.

5. Individuals taking medications which might interfere with the test results including the use of steroidal/non-steroidal anti-inflammatory drugs or antihistamines, acutane or any type of prescription acne medication.

6. Individuals who have undergone a laser resurfacing or dermabrasion procedure on the face in the past 2 years or a chemical face peel (deep peel in the past 1 year; superficial peel in the past two months).

7. Individuals with acne, active atopic dermatitis/eczema or psoriasis.

8. Individuals who have had a surgical “cosmetic” procedure on the face within the past 10 years.

9. Treatment or history of any type of cancer.

10. Individuals who are currently under treatment for asthma or diabetes.

11. Individuals with a known sensitivity to cosmetics or personal care products.

3.2 Test Procedure

The study was designed as a 4-week study in which a test product prepared according to example 1 was used by each of the test panelists according to instructions. Subjects were directed to use the product twice a day (AM and PM) by shaking the bottle well. Pressing the actuator button to dispense small amount onto fingertips and massage into skin.

3.2.1 Baseline Visit

In a baseline visit, a trained technician visually evaluated crow's feet fine lines and wrinkles to determine qualification. A Cutometer® reading was taken to measure skin firmness and skin elasticity and a Corneometer® measurement was taken to measure skin hydration. Digital photographs were taken with the Visia CR® Imaging System (Canfield Scientific, Fairfield, N.J.). Using ImagePro® software (MediaCybernetics, Bethesda, Md.), the images were analyzed to determine changes in crow's feet fine lines and crow's feet wrinkles.

Additionally, an irritation evaluation was conducted for safety purposes. Subjects made the first application of the test product under the supervision of a trained technician. Thirty (30) minutes following application, Cutometer® and Corneometer® measurements and digital photographs were repeated and subjects completed a questionnaire. Subjects returned following 1 and 4 weeks of product use for Cutometer® and Corneometer® measurements, photographs and to complete additional questionnaires. Evaluations of efficacy was based on a comparison of baseline vs. each observation period.

Subjects also received the test product and a daily diary with the following use instructions:

INSTRUCTIONS: The following items must be included in this diary:

1. Date and time test article (a.m. and p.m.) was used.

2. Any comments or observations you may have had while using the test article.

3.2.2. One-Use, One- and Four-Week Evaluation

Subjects were evaluated after one use of the product and after 1 and 4 weeks of product use for additional Cutometer® and Corneometer® measurements, digital photographs and an irritation evaluation. Additionally, subjects were required to complete a questionnaire at each evaluation.

3.3 Clinical Evaluation Procedures

Evaluations were conducted in accordance with the following scales and procedures.

3.3.1 Evaluation of Crow's Feet Fine Lines and Wrinkles

At the baseline visit only (to determine qualification), a trained technician evaluated crow's feet fine lines and wrinkles on the face of each subject according to the scale below. Scale for Scoring Fine Lines and Wrinkles:

0=None

1-3=Slight

4-6=Noticeable

7-9=Very Noticeable

3.3.2 Cutometer® Measurement

At each visit, the firmness of the skin was measured on the face of each subject using the Cutometer®. The R0 and F4 parameters were used to determine changes in skin firmness and the R2 parameter was used to determine changes in skin elasticity. For the R0 and F4 parameters, a decrease in Cutometer® measurements indicated an improvement (increase) in skin firmness. An increase represented a worsening. For the R2 parameter, an increase in Cutometer® measurements indicated an improvement (increase) in skin firmness. A decrease represented a worsening.

3.3.3 Corneometer® Measurement

At each visit, the moisture content of the skin was measured on the face of each subject using the Corneometer®. An increase in Corneometer® measurements indicated an improvement (increase) in skin hydration. A decrease represented a worsening.

3.3.4 Irritation Evaluation

At each visit, the following scale was used to assess irritation on the face of each subject. This evaluation was for safety purposes only and was not used in determining efficacy. Assessment Scale for Irritation:

0=No evidence of any effect

+=Barely perceptible irritation present

1=Mild irritation present

2=Moderate irritation present

3=Marked irritation present

4=Severe irritation present

3.3.5 Digital Photography Procedure and Analysis

At each visit, digital images of the face of each subject were taken from the front, right and left views using the Visia CR® 2.2 (Canfield Scientific, Fairfield, N.J.). In order to ensure consistency between the photographs, each subject was draped with a black cloth around the shoulders in order to eliminate the appearance of clothing in the pictures and each subject wore a black headband to pull hair off of and away from the face. The images were analyzed using Image Pro® software (MediaCybernetics, Bethesda, Md.) to determine changes (if any) in:

Crow's feet fine lines

Crow's feet wrinkles

3.3.5.1 Crow's Feet Fine Lines—Image Analysis

In order to determine changes in crow's feet fine lines, each digital image was scanned horizontally and vertically to collect the red, green and blue intensities of the pixels. The proprietary mathematical algorithm in Visia CR® uses the pixel intensities of the scanned areas to calculate the texture score based on the totals of the mean intensities of the red, green and blue pixels. Texture scores are a single number calculated based on skin features. A decrease in the texture score represented an improvement (or decrease) in the appearance of crow's feet fine lines.

3.3.5.2 Crow's Feet Wrinkles—Image Analysis

In order to determine changes in crow's feet wrinkles, each digital image was scanned horizontally and vertically to collect the red, green and blue intensities of the pixels. The proprietary mathematical algorithm in Visia CR® uses the pixel intensities of the scanned areas to calculate the texture score based on the totals of the mean intensities of the red, green and blue pixels. Texture scores are a single number calculated based on skin features. A decrease in the texture score represented an improvement (or decrease) in the appearance of crow's feet wrinkles.

### 4.0 Results and Discussion

A total of thirty-two (32) female subjects between the ages of 51 and 65 years, were empanelled. (Due to an instrument malfunction, baseline readings were unable to be taken on 6 of the original subjects, who were replaced by 6 additional subjects meeting the study criteria.

4.1 Crow's Feet Fine Lines—Image Analysis

At baseline, thirty (30) minutes following a single application of the product and after 1 and 4 weeks of product use, a trained technician took digital images of the face of each subject with the Visia CR® imaging system. Using ImagePro® software, the images were analyzed to determine changes (if any) in crow's feet fine lines. Table 6 presents a summary of the crow's feet fine lines image analysis.

TABLE 6
Crow's Feet Fine Lines - Image Analysis

| | | | | % of Subjects |
|---|---|---|---|---|
| | | | Mean % | with Improve- |
| | Mean Score ± | p- | Change from | ment from |
| | S.D | value | Baseline | Baseline |
| Baseline | 504.9 ± 126.5 | — | — | — |
| 30 Mins | 449.6* ± 66.6 | <0.001 | −11.0% | 94% |
| Post-Appl. | | | | |
| 1 Week | 420.7* ± 50.5 | <0.001 | −16.7% | 100% |
| 4 Weeks | 357.0* ± 34.5 | <0.001 | −29.3% | 100% |

*Statistically significant when compared with baseline, p < 0.05

When scores taken 30 minutes following a single application of the product and after 1 and 4 weeks of product use were compared with baseline, there were mean percent improvements of 11.0%, 16.7% and 29.3%, respectively, based on image analysis. The improvements observed were highly significant when compared with baseline. A total of 94%, 100% and 100% of the subjects showed improvement 30 minutes following a single application of the product and after 1 and 4 weeks of product use, respectively.

4.2 Crow's Feet Wrinkles—Image Analysis

At baseline, thirty (30) minutes following a single application of the product and after 1 and 4 weeks of product use, a trained technician took digital images of the face of each subject with the Visia CR® imaging system. Using ImagePro® software, the images were analyzed to determine changes (if any) in crow's feet wrinkles. Table 7 presents a summary of the crow's feet wrinkles image analysis.

TABLE 7
Crow's Feet Wrinkles - Image Analysis

| | | | | % of Subjects |
|---|---|---|---|---|
| | | | Mean % | with Improve- |
| | Mean Score ± | p- | Change from | ment from |
| | S.D | value | Baseline | Baseline |
| Baseline | 908.4 ± 306.3 | — | — | — |
| 30 Mins | 760.3* ± 185.7 | <0.001 | −16.3% | 94% |
| Post-Appl. | | | | |
| 1 Week | 713.8* ± 127.5 | <0.001 | −21.4% | 86% |
| 4 Weeks | 582.8* ± 83.7 | <0.001 | −35.8% | 100% |

*Statistically significant when compared with baseline, p < 0.05

When scores taken 30 minutes following a single application of the product and after 1 and 4 weeks of product use were compared with baseline, there were mean percent improvements of 16.3%, 21.4% and 35.8%, respectively, based on image analysis. The improvements observed were highly significant when compared with baseline. A total of 94%, 86% and 100% of the subjects showed improvement 30 minutes following a single application of the product and after 1 and 4 weeks of product use, respectively.

4.3 Skin Hydration—Corneometer® Measurements

At baseline, thirty (30) minutes following a single application of the product and after 1 and 4 weeks of product use, a trained technician measured the moisture content of the skin on the face of each subject using the Corneometer®.

Table 8 presents a summary of the Corneometer® measurements.

TABLE 8
Skin Hydration - Corneometer ® Measurements

| | | | | % of Subjects |
|---|---|---|---|---|
| | | | Mean % | with Improve- |
| | Mean Score ± | p- | Change from | ment from |
| | S.D | value | Baseline | Baseline |
| Baseline | 38.7 ± 9.8 | — | — | — |
| 30 Mins | 59.3* ± 12.1 | <0.001 | 53.2% | 100% |
| Post-Appl. | | | | |
| 1 Week | 59.2* ± 13.9 | <0.001 | 53.0% | 100% |
| 4 Weeks | 62.6* ± 14.3 | <0.001 | 61.8% | 97% |

*Statistically significant when compared with baseline, p < 0.05

When scores taken 30 minutes following a single application of the product and after 1 and 4 weeks of product use were compared with baseline, there were mean percent improvements of 53.2%, 53.0% and 61.8%, respectively, based on Corneometer® measurements. The improvements observed were highly significant when compared with baseline. A total of 100%, 100% and 97% of the subjects showed improvement 30 minutes following a single application of the product and after 1 and 4 weeks of product use, respectively.

4.4 Skin Firmness/Tightness—Cutometer® R0 Measurements

At baseline, thirty (30) minutes following a single application of the product and after 1 and 4 weeks of product use, a trained technician measured skin firmness/tightness on the face of each subject using the Cutometer® R0 parameter. Table 9 presents a summary of the Cutometer® R0 measurements.

TABLE 9
Skin Firmness/Tightness - Cutometer ® R0 Measurements

| | | | | % of Subjects |
|---|---|---|---|---|
| | | | Mean % | with Improve- |
| | Mean Score ± | p- | Change from | ment from |
| | S.D | value | Baseline | Baseline |
| Baseline | 0.092 ± 0.096 | — | — | — |
| 30 Mins | 0.086 ± 0.051 | 0.385 | −6.5% | 43% |
| Post-Appl. | | | | |
| 1 Week | 0.125* ± 0.025 | <0.001 | 35.9% | 13% |
| 4 Weeks | 0.194* ± 0.121 | <0.001 | 110.9% | 23% |

*Statistically significant when compared with baseline, p < 0.05

When scores taken 30 minutes following a single application of the product and after 1 and 4 weeks of product use were compared with baseline, there was a mean percent improvement of 6.5% and mean percent worsening of 35.9% and 110.9%, respectively, based on Cutometer® R0 measurements. The changes observed after 1 and 4 weeks of product use were significant when compared with baseline. A total of 43%, 13% and 23% of the subjects showed improvement 30 minutes following a single application of the product and after 1 and 4 weeks of product use, respectively.

4.5 Skin Firmness/Tightness—Cutometer® F4 Measurements

At baseline, thirty (30) minutes following a single application of the product and after 1 and 4 weeks of product use, a trained technician measured skin firmness/tightness on the face of each subject using the Cutometer® F4 parameter. Table 10 presents a summary of the Cutometer® F4 measurements.

TABLE 10
Skin Firmness/Tightness - Cutometer ® F4 Measurements

| | | | | % of Subjects |
|---|---|---|---|---|
| | | | Mean % | with Improve- |
| | Mean Score ± | p- | Change from | ment from |
| | S.D | value | Baseline | Baseline |
| Baseline | 1.519 ± 0.679 | — | — | — |
| 30 Mins | 1.723 ± 1.013 | 0.243 | 13.4% | 43% |
| Post-Appl. | | | | |
| 1 Week | 2.611* ± 0.513 | <0.001 | 71.9% | 7% |
| 4 Weeks | 4.344* ± 2.772 | <0.001 | 186.0% | 17% |

*Statistically significant when compared with baseline, p < 0.05

When scores taken 30 minutes following a single application of the product and after 1 and 4 weeks of product use were compared with baseline, there were mean percent worsenings of 13.4%, 71.9% and 186.0%, respectively, based on Cutometer® F4 measurements. The changes observed after 1 and 4 weeks of product use were significant when compared with baseline. A total of 43%, 7% and 17% of the subjects showed improvement 30 minutes following a single application of the product and after 1 and 4 weeks of product use, respectively.

4.6 Skin Elasticity—Cutometer® R2 Measurements

At baseline, thirty (30) minutes following a single application of the product and after 1 and 4 weeks of product use, a trained technician measured skin elasticity on the face of each subject using the Cutometer® R2 parameter. Table 11 presents a summary of the Cutometer® R2 measurements.

TABLE 11
Skin Elasticity - Cutometer ® R2 Measurements

| | | | | % of Subjects |
|---|---|---|---|---|
| | | | Mean % | with Improve- |
| | Mean Score + | p- | Change from | ment from |
| | S.D | value | Baseline | Baseline |
| Baseline | 0.333 ± 0.107 | — | — | — |
| 30 Mins | 0.523* ± 0.177 | <0.001 | 57.1% | 100% |
| Post-Appl. | | | | |
| 1 Week | 0.559* ± 0.132 | <0.001 | 67.9% | 100% |
| 4 Weeks | 0.683* ± 0.136 | <0.001 | 105.1% | 97% |

*Statistically significant when compared with baseline, p < 0.05

When scores taken 30 minutes following a single application of the product and after 1 and 4 weeks of product use were compared with baseline, there were mean percent improvements of 57.1%, 67.9% and 105.1%, respectively, based on Cutometer® R2 measurements. The improvements observed were highly significant when compared with baseline. A total of 100%, 100% and 97% of the subjects showed improvement 30 minutes following a single application of the product and after 1 and 4 weeks of product use.

4.7 Irritation Evaluation—Technician Evaluation

At baseline, thirty (30) minutes following a single application of the product and after 1 and 4 weeks of product use, a trained technician evaluated the face of each subject for irritation. Table 12 presents a summary of the technician irritation evaluation.

TABLE 12
Irritation Evaluation - Technician Evaluation

| | | | % of Subjects |
|---|---|---|---|
| | | Mean % | with Improve- |
| | | Change from | ment from |
| | Mean Score | Baseline | Baseline |
| Baseline | 0 | — | — |
| 30 Mins | 0 | 0% | 0% |
| Post-Appl. | | | |
| 1 Week | 0 | 0% | 0% |
| 4 Weeks | 0 | 0% | 0% |

There was no irritation observed on the any subject during the course of the study.

4.8 Thirty-Minute Post-Application Questionnaire—Response Summary

Thirty (30) minutes following a single application of the product subjects were required to complete a questionnaire Table 13 presents a summary of the questionnaire responses.

TABLE 13
Thirty-Minute Post-Application Questionnaire --
Subject Response Summary

| | Strongly | No | Disagree |
|---|---|---|---|
| | Agree | Opin- | to Strongly |
| | to Agree | ion | Disagree |
| This product immediately reduced the | 27% | 50% | 23% |
| appearance of fine lines. | | | |
| This product visibly reduced the | 33% | 53% | 14% |
| appearance of fine lines. | | | |
| This product significantly reduced | 33% | 50% | 17% |
| the appearance of fine lines. | | | |
| This product immediately reduced | 30% | 53% | 17% |
| the appearance of deep wrinkles. | | | |
| This product visibly reduced the | 36% | 43% | 21% |
| appearance of deep wrinkles. | | | |
| This product significantly reduced | 36% | 43% | 21% |
| the appearance of deep wrinkles. | | | |
| This product reduced the appearance | 36% | 40% | 24% |
| of forehead wrinkles. | | | |
| This product reduced the appearance | 33% | 43% | 24% |
| of “eleven lines” (between brows). | | | |
| This product reduced the appearance | 33% | 47% | 20% |
| of marionette lines (vertical lines | | | |
| from lip to chin). | | | |
| This product reduced the appearance | 30% | 47% | 23% |
| of “parentheses lines” (vertical | | | |
| lines from nose to chin). | | | |
| This product reduced the appearance | 43% | 40% | 17% |
| of lines around the eye area. | | | |
| This product visibly improved the | 47% | 23% | 20% |
| appearance of skin firmness. | | | |
| This product significantly improved | 43% | 37% | 20% |
| the appearance of skin firmness. | | | |
| This product visibly improved the | 36% | 43% | 21% |
| appearance of skin elasticity. | | | |
| This product significantly improved | 30% | 50% | 20% |
| the appearance of skin elasticity. | | | |
| This product instantly hydrated the | 66% | 27% | 7% |
| skin. | | | |
| This product significantly hydrated | 67% | 23% | 10% |
| the skin. | | | |
| This product improved the appearance | 60% | 27% | 13% |
| of skin texture. | | | |
| My skin looks smoother. | 67% | 23% | 10% |
| My skin looked significantly smoother. | 60% | 27% | 13% |
| My skin looked younger. | 40% | 47% | 13% |
| My skin looked significantly younger. | 33% | 53% | 14% |

4.9 Week 1 Questionnaire—Response Summary

After 1 week of product use, subjects were required to complete a questionnaire.

Table 14 presents a summary of the questionnaire responses.

TABLE 14
Week 1 Questionnaire -- Subject Response Summary

| | Strongly | No | Disagree |
|---|---|---|---|
| | Agree | Opin- | to Strongly |
| | to Agree | ion | Disagree |
| This product immediately reduced the | 64% | 20% | 16% |
| appearance of fine lines. | | | |
| This product visibly reduced the | 80% | 13% | 7% |
| appearance of fine lines. | | | |
| This product significantly reduced | 73% | 17% | 10% |
| the appearance of fine lines. | | | |
| This product immediately reduced | 60% | 17% | 23% |
| the appearance of deep wrinkles. | | | |
| This product visibly reduced the | 74% | 10% | 16% |
| appearance of deep wrinkles. | | | |
| This product significantly reduced | 57% | 20% | 23% |
| the appearance of deep wrinkles. | | | |
| This product reduced the appearance | 80% | 13% | 7% |
| of forehead wrinkles. | | | |
| This product reduced the appearance | 80% | 7% | 13% |
| of “eleven lines” (between brows). | | | |
| This product reduced the appearance | 64% | 23% | 13% |
| of marionette lines (vertical lines | | | |
| from lip to chin). | | | |
| This product reduced the appearance | 56% | 27% | 17% |
| of “parentheses lines” (vertical | | | |
| lines from nose to chin). | | | |
| This product reduced the appearance | 83% | 7% | 10% |
| of lines around the eye area. | | | |
| This product visibly improved the | 90% | 7% | 3% |
| appearance of skin firmness. | | | |
| This product significantly improved | 83% | 10% | 7% |
| the appearance of skin firmness. | | | |
| This product visibly improved the | 90% | 3% | 7% |
| appearance of skin elasticity. | | | |
| This product significantly improved | 77% | 13% | 10% |
| the appearance of skin elasticity. | | | |
| This product instantly hydrated the | 93% | 7% | 0% |
| skin. | | | |
| This product significantly hydrated | 87% | 13% | 3% |
| the skin. | | | |
| This product improved the appearance | 93% | 7% | 0% |
| of skin texture. | | | |
| My skin looks smoother. | 97% | 3% | 0% |
| My skin looked significantly smoother. | 87% | 13% | 0% |
| My skin looked younger. | 76% | 17% | 7% |
| My skin looked significantly younger. | 63% | 27% | 10% |

4.10 Week 4 Questionnaire—Response Summary

After 1 week of product use, subjects were required to complete a questionnaire. Table 15 presents a summary of the questionnaire responses.

TABLE 15
Week 4 Questionnaire -- Subject Response Summary

| | Strongly | No | Disagree |
|---|---|---|---|
| | Agree | Opin- | to Strongly |
| | to Agree | ion | Disagree |
| This product immediately reduced the | 67% | 10% | 23% |
| appearance of fine lines. | | | |
| This product visibly reduced the | 83% | 10% | 7% |
| appearance of fine lines. | | | |
| This product significantly reduced | 77% | 10% | 13% |
| the appearance of fine lines. | | | |
| This product immediately reduced | 57% | 13% | 30% |
| the appearance of deep wrinkles. | | | |
| This product visibly reduced the | 67% | 13% | 20% |
| appearance of deep wrinkles. | | | |
| This product significantly reduced | 70% | 7% | 23% |
| the appearance of deep wrinkles. | | | |
| This product reduced the appearance | 76% | 17% | 7% |
| of forehead wrinkles. | | | |
| This product reduced the appearance | 73% | 10% | 17% |
| of “eleven lines” (between brows). | | | |
| This product reduced the appearance | 67% | 13% | 20% |
| of marionette lines (vertical lines | | | |
| from lip to chin). | | | |
| This product reduced the appearance | 63% | 17% | 20% |
| of “parentheses lines” (vertical | | | |
| lines from nose to chin). | | | |
| This product reduced the appearance | 80% | 10% | 10% |
| of lines around the eye area. | | | |
| This product visibly improved the | 90% | 7% | 3% |
| appearance of skin firmness. | | | |
| This product significantly improved | 77% | 13% | 10% |
| the appearance of skin firmness. | | | |
| This product visibly improved the | 90% | 7% | 3% |
| appearance of skin elasticity. | | | |
| This product significantly improved | 83% | 10% | 7% |
| the appearance of skin elasticity. | | | |
| This product instantly hydrated the | 97% | 0% | 0% |
| skin. | | | |
| This product significantly hydrated | 90% | 10% | 0% |
| the skin. | | | |
| This product improved the appearance | 97% | 3% | 0% |
| of skin texture. | | | |
| My skin looks smoother. | 100% | 0% | 0% |
| My skin looked significantly smoother. | 97% | 3% | 0% |
| My skin looked younger. | 74% | 23% | 3% |
| My skin looked significantly younger. | 63% | 30% | 7% |

### 5.0 Conclusions

A clinical efficacy study was conducted with 32 subjects to determine if Test Article: Dermal Quench Wrinkle Warrior improved skin firmness, skin elasticity, skin hydration, the appearance of crow's feet fine lines and the appearance of crow's feet wrinkles 30 minutes following a single application and after 1 and 4 weeks of twice daily use. Crow's feet fine lines were significantly improved 30 minutes following a single application and after 1 and 4 weeks of use, based on image analysis. Crow's feet wrinkles were significantly improved 30 minutes following a single application and after 1 and 4 weeks of use, based on image analysis. Skin hydration was significantly improved 30 minutes following a single application and after 1 and 4 weeks of use, based on Corneometer® measurements. Skin elasticity was significantly improved 30 minutes following a single application and after 1 and 4 weeks of use, based on Cutometer® R2 measurements. There was no irritation observed on any subject during the course of the study. After 1 and 4 weeks of product use, the product was met with a moderate to high level of subject acceptance.

From the forgoing description, one skilled in the art can easily ascertain the essential characteristics of this invention, and without departing from the spirit and scope thereof, can make changes and modifications of the invention to adapt it to various usage and conditions and to utilize the present invention to its fullest extent. The preceding preferred specific embodiments are to be construed as merely illustrative, and not limiting of the scope of the invention in any way whatsoever.

1. A cosmeceutical formulation for treating damaged skin comprising:
a first hyaluronic acid having a molecular weight of from about 25 kDa to about 100 kDa,
a second hyaluronic acid having a molecular weight of from about 1 kDa to about 20 kDa, and
a polyglutamate.

2. The cosmeceutical formulation of claim 1, comprising
from about 0.001 wt % to about 0.5 wt % of the first hyaluronic acid,
from about 0.001 wt % to about 0.5 wt % of the second hyaluronic acid, and
from about 0.001 wt % to about 5 wt % of the polyglutamate.

3. The cosmeceutical formulation of claim 1, further comprising a third hyaluronic acid having a molecular weight of greater than about 500 kDa.

4. The cosmeceutical formulation of claim 3, comprising
from about 0.0001 wt % to about 5 wt % of the third hyaluronic acid.

5. The cosmeceutical formulation of claim 1, wherein the first hyaluronic acid is a hydrolyzed hyaluronic acid having a molecular weight of about 50 kDa.

6. The cosmeceutical formulation of claim 1, wherein the second hyaluronic acid is a hydrolyzed hyaluronic acid having a molecular weight of less than about 10 kDa.

7. The cosmeceutical formulation of claim 1, wherein the second hyaluronic acid has a molecular weight of from about 3 kDa to about 10 kDa.

8. The cosmeceutical formulation of claim 1, wherein the polyglutamate has a molecular weight of from about 1000 kDa to about 5000 kDa.

9. The cosmeceutical formulation of claim 1, further comprising about 1.0-10 wt % of one or more perfluorocarbons (PFC's).

10. The cosmeceutical formulation of claim 1, further comprising one or more of niacin, vitamin E, vitamin C, other vitamins, minerals, enzymes, amino acids, antioxidants, or natural fragrances.

11. The cosmeceutical formulation of claim 1, further comprising one or more of: emulsifiers; rheology modifiers; humectants; surfactants; emollients; pH modifiers; antimicrobial agents; colorants; aromas; or antioxidants.

12. The cosmeceutical formulation of claim 1, wherein the formulation is for treatment of aging skin, wrinkled skin, or other damage to the skin.

13. The cosmeceutical formulation of claim 1, wherein the cosmeceutical formulation is aqueous or an oil and water emulsion.

14. A method for treating damaged or wrinkled skin, comprising administering the cosmeceutical formulation according to claim 1.

15. The method of claim 14, wherein the cosmeceutical formulation is administered at least once daily.

16. The method of claim 15, wherein the cosmeceutical formulation is administered for at least one week.

17. The method of claim 14, wherein the damaged or wrinkled skin consists of one or more of crow's feet fine lines, crow's feet wrinkles, fine lines, deep wrinkles, forehead wrinkles, eleven lines, marionette lines, parentheses lines, and eye area lines.

18. The method of claim 14, wherein the administration improves one or more of skin hydration, tightness, firmness, elasticity, texture, and smoothness.

19. A method for improving skin comprising administering the cosmeceutical formulation according to claim 1.

20. The method of claim 19, wherein improving the skin comprises reducing one or more of crow's feet fine lines, crow's feet wrinkles, fine lines, deep wrinkles, forehead wrinkles, eleven lines, marionette lines, parentheses lines, and eye area lines.

21. The method of claim 19, wherein improving the skin comprises improving one or more of skin hydration, tightness, firmness, elasticity, texture, and smoothness.
